(19) United States
(12) Patent Application Publication
(10) Pub. No.: US 2022/0311739 A1
(43) Pub. Date: Sep. 29, 2022

(54) COMPUTER HARDWARE FOR A COMPUTER-CONTROLLED MEDICAL DEVICE AND METHOD FOR CONTROLLING A COMPUTER-CONTROLLED MEDICAL DEVICE

(71) Applicant: Carl Zeiss Meditec AG, Jena (DE)

(72) Inventors: Lars Fiedler, Jena (DE); Jens Bojko, Jena (DE)

(73) Assignee: Carl Zeiss Meditec AG, Jena (DE)

(21) Appl. No.: 17/641,505

(22) PCT Filed: Sep. 8, 2020
(86) PCT No.: PCT/EP2020/075037

§ 371 Date: Mar. 9, 2022

(30) Foreign Application Priority Data
Sep. 10, 2019 (DE) 10 2019 213 707.5

(51) Int. Cl. H04L 9/40; G06F 8/65; G06F 1/3287; G16H 40/67; H04L 12/10

(52) U.S. Cl. CPC H04L 63/02; G06F 8/65; G06F 1/3287; G16H 40/67; H04L 12/10; H04L 67/12

(57) ABSTRACT

Computer hardware for a computer-controlled medical device, a computer-controlled medical device and a method of controlling a computer-controlled medical device. Computer hardware is configured such that there is no possibility of medically relevant software being manipulated from outside. The computer includes a control hardware module, serving an internal network of the medical device, and a gate control hardware module, serving a communication network, wherein the control hardware module and the gate control hardware module are arranged such that the communication network and the internal network are completely separated, and the gate control hardware module forms a security moderator between the communication network and the internal network. A method operates the medically relevant software in an internal network, which is completely separate from a gate control by which the medical device communicates with an external communication network and secures the medical device with regard to the external communication network.

### RELATED APPLICATIONS

This application is a National Phase entry of PCT Application No. PCT/EP2020/075037 filed Sep. 8, 2020, which application claims the benefit of priority to DE Application No. 10 2019 213 707.5 filed Sep. 10, 2019, the entire disclosures of which are incorporated herein by reference.

### TECHNICAL FIELD

The present invention relates to computer hardware for a computer-controlled medical apparatus, a computer-controlled medical apparatus and a method for controlling a computer-controlled medical apparatus.

### BACKGROUND

Modern medical solutions require connectivity. To this end, these medical apparatuses need to be networked. Networking of such systems is possible from a technical point of view (typically LAN/WAN/WLAN etc.), but this results in the risk of manipulation of functions or data on the medical apparatuses. To minimize these risks it is necessary to secure the apparatus interfaces, which need to be updated regularly on account of persistent novel external threats. As a rule, the operating system of the respective apparatus is updated in this context. However, the operating system of a medical apparatus also forms the basis of the medically relevant software, and so modifications here require a comprehensive reverification of the entire software of the medical apparatus. It may even be the case that a newer version of the operating system no longer supports the existing computer hardware so that the hardware of the medical apparatus also needs to be modified. The replacement of components with a relatively large power consumption is relevant to the certification (list of critical components), and so great outlay may arise quickly in this case.

These days, providing only general protection for a communications network, for example a clinic communications network, but operating a medical apparatus 100A using a control computer 300A with medically relevant software 600A without protection against possible attacks from the connected clinic communications network 500A, as shown in FIG. 1a, is hardly possible. The recent, frequent hacker attacks on clinic communications networks 500A highlight the necessity to equip medical apparatuses 100A with protection for the internal apparatus functionality. There are also regulatory demands for specific protection mechanisms, including network security.

A firewall 700B as a possible security system in front of the medical apparatus 100B itself, or specifically in front of the control computer 300B thereof with the medically relevant software 600B thereof, as illustrated in FIG. 1b, restricts the network access to the clinic communications network 500B on the basis of fixed rules for data traffic. Security holes of the services behind said firewall remain, however. If the security holes are exploited or the firewall 700B is overcome, direct access to the control computer 300B of the medical apparatus 100B is possible.

Attacks on the control computer 300C of a medical apparatus 100C, on which both the medically relevant apparatus software 600C and the firewall 700C are run, as shown in FIG. 1c, have greater opportunities to manipulate and circumvent the protection due to software errors of the firewall 700C and also of other services, than would be the case for separate solutions. Hence, potential attackers would be granted direct access to the control computer 700C of the medical apparatus 100C. Updates to the firewall 700C and to the operating system of the control computer 300C are still necessary and may also lead to incompatibility with existing hardware in this case.

### SUMMARY OF THE INVENTION

Embodiments of the present invention configure computer hardware for a computer-controlled medical apparatus so that a manipulation of medically relevant software, which manipulation is controlled from outside of the medical apparatus, in particular from an external communications network, is precluded. Moreover, the intention is to describe a corresponding method for controlling a computer-controlled medical apparatus.

Computer hardware according to the invention for a computer-controlled medical apparatus comprises a control hardware module serving an internal network of the medical apparatus and a gate control hardware module serving a communications network. Here, the control hardware module and the gate control hardware module are disposed such that there is a complete logical and physical separation of communications network and internal network of the medical apparatus, and the gate control hardware module forms a security entity between the communications network and the internal network of the medical apparatus. Thus, the gate control hardware module does not only act as a type of “switching module” which may optionally also be “deactivated” or circumvented if desired by the operator and which could thus clear a direct path between the communications network and the internal network. Rather, it is the only path between communications network and internal network, whose controlling and blocking function in respect of direct access from the communications network cannot be “deactivated” or otherwise circumvented without a request from the internal network in this respect. This security entity renders automatic or even only inadvertent access to the control hardware module, and hence also access to the internal network of the medical apparatus, impossible: It ensures that data, updates and other queries coming from the external communications network only reach the internal network, or data from the internal network are only output to the external communications network, following an explicit request (by the operator or the medical apparatus itself), for example by virtue of an operator triggering this by multiple active confirmations or by virtue of the medical apparatus itself starting such a procedure from its internal network.

In particular, the communications network is a clinic communications network. This may be a communications network that is completely separate from a public communications network, or else a communications network that also offers access to a general, public communications network, said access in turn being able to be direct or else being able to be secured vis-à-vis the public communications network by use of at least one additional control entity. In this context, a communications network contains a network which not only makes data available, queries data and transmits data, but which also makes control commands available, queries control commands and transmits control commands.

The internal network of the medical apparatus comprises the control of all components of the medical apparatus which are required for its medical use. In this context, the term “control” contains both the control of all relevant components of the medical apparatus (for example, controlling the laser, the scanners, the optical unit and also integrated examination devices in the case of an ophthalmological laser therapy system) and also data acquisition, data processing and data output before, during or after the use of the medical apparatus.

To control all components of the medical apparatus, the control hardware module may comprise a single central module serving all components. In this case, the internal network of the medical apparatus is an ideal network on the control hardware module. However, the control hardware module for example comprises a plurality of control hardware module components, which may also be spatially separated from one another and which communicate among one another, such that the internal network of the medical apparatus is a real network in this case.

Thus, there is segregation between the medically relevant region of the medical apparatus with the correspondingly relevant software for calling the functions of the medical apparatus and the region of the medical apparatus containing the functionalities for connectivity, that is to say the communication between the medical apparatus and the clinic communications network. As a result, the gate control hardware module protects apparatus interfaces of the medical apparatus from external attacks.

The gate control hardware module thus represents a security entity of the medical apparatus, which can easily be supplied with security updates and which can easily be replaced without requiring complete verification of the medically relevant hardware and software of the medical apparatus.

The computer hardware is for example configured to operate the control of the gate control hardware module exclusively from the internal network of the medical apparatus.

While all communications network services are realized on the gate control hardware module, the latter itself is only controlled from the internal network of the medical apparatus by operation of the control hardware module, preventing corruption by external manipulation, for example by an unauthorized intrusion in the medical apparatus via the communications network.

Computer hardware is for example configured to forward the required data to the internal network of the medical apparatus via proprietary protocols.

Thus, there is no network gateway, via which data could reach the internal network of the medical apparatus in uncontrolled fashion. However, variations in the protocol between the external communications network and the internal network of the medical apparatus, that is to say variations of the protocol or interface between the gate control hardware module and the control hardware module of the medical apparatus, are possible in this context, for example Ethernet, CAN, SPI, I2C, RS485, RS232.

In one embodiment of the computer hardware according to the invention, the power consumption of the gate control hardware module is limited. For example in this context, the power consumption of the gate control hardware module is less than 15 W in the case of a current intensity of less than 7 A.

As a result, changes to the gate control hardware module become irrelevant from a regulatory and/or standards point of view.

Computer hardware according to the invention for a computer-controlled medical apparatus, configured to apply security updates only to the software of the gate control hardware module, is for example particularly advantageous.

A security update for the software, including the operating system, is therefore easily implementable without being a risk to the medically relevant software on the control hardware module serving the internal network of the medical apparatus.

This allows use of an operating system which comprises regular security updates and/or long-term support.

Moreover, this allows variations of the utilized operating system of the gate control hardware module as a matter of principle.

Additionally, in an embodiment of the computer hardware according to the invention, a firewall is implemented in hardware and/or software.

A firewall for further increasing the protection of the medical apparatus against external attacks or else only against externally introduced malfunctions may be provided as an additional hardware module between the gate control hardware module and the control hardware module, or between the communications network and the gate control hardware module. However, a firewall may also be realized as software in a gate control hardware module or in a control hardware module.

Computer hardware according to the invention which is configured to secure data-transferring interfaces of the medical apparatus by virtue of data services being provided by the gate control hardware module is for example advantageous.

By way of example, such interfaces can be USB interfaces.

Furthermore, computer hardware for a computer-controlled medical apparatus, which computer hardware is configured to derive the power supply of the gate control hardware module from the power supply of the medical apparatus, is for example advantageous.

By way of example, this may be implemented by using Power over Ethernet (PoE). This prevents the gate control hardware module from being able to be operated without the medical apparatus being in operation. This is a further measure for further reducing the risk of manipulation of the medical apparatus.

A further measure for preventing manipulations of the medical apparatus consists in the minimization of network services provided by the gate control software on the gate control hardware module. Exchange of data of the medical apparatus via the communications network is thus facilitated but restricted to the strictly necessary minimum.

An alternative solution to a gate control hardware module integrated into the medical apparatus is offered by computer hardware in which the gate control hardware module is disposed outside of the medical apparatus and comprises a mechanical protection for the connection to the control hardware module of the medical apparatus. This mechanical protection is designed such that it renders unnoticed “disconnection” of the gate control hardware module impossible. Moreover, a manipulation of the medical apparatus by intermittent unnoticed “bridging” of the gate control hardware module is for example precluded by virtue of rendering a restart of the medical apparatus impossible when the mechanical protection of the connection between gate control hardware module and control hardware module has been separated or in the case of an intermittent “disappearance” of the gate control hardware module.

Embodiments of the invention include a computer-controlled medical apparatus having computer hardware according to the invention as described above. In particular, it is achieved by an ophthalmological laser therapy system containing such computer hardware according to the invention.

In a method according to the invention for controlling a computer-controlled medical apparatus, the medically relevant software is exclusively operated in an internal network of the medical apparatus. The medical apparatus communicates with an external communications network exclusively by operation of a gate control. This gate control secures the medical apparatus vis-à-vis the external communications network and secures it so that no data traffic is admitted between internal network and gate control, and hence also no data traffic being admitted to the external communications network, until there is a request from the internal network or a qualified manual input by an operator. As a result, the gate control implements a complete logical and physical separation of communications network and internal network of the medical apparatus.

In this context, a request from the internal network may be implemented by way of routines established therein, while a qualified manual input is only implemented using operator-identifying applications and should for example be implemented using multiple password protection.

Thus, the function of controlling the components of the medical apparatus, and hence the medically relevant processes of the medical apparatus, is segregated from the connectivity function to an external communications network and the protection vis-à-vis this external network. Apparatus interfaces of the medical apparatus are protected from external attacks by way of the gate control.

This gate control cannot be “deactivated” or circumvented for example, even if desired by the operator, and thus clear a direct path between the communications network and the internal network. Direct access from the communications network without a request to this end from the internal network is therefore always blocked and there are no circumstances under which this block can be circumvented since there is no option in this respect for a “deactivation” or modification in relation to direct access. Although this restricts the operator's freedoms, this also prevents inadvertent interventions by the operator that could facilitate direct access from the communications network, thus increasing the security of the medical apparatus in relation to preventing an inadvertent or deliberate manipulation of the medical apparatus from the communications network.

It is particularly advantageous if, in such a method according to an example embodiment of the invention, the control of the gate control is implemented exclusively from the internal network of the medical apparatus.

It is further advantageous if, in such a method according to an example embodiment of the invention, data required by the medical apparatus are transmitted to the internal network of the medical apparatus via proprietary protocols.

It is also expedient if, in a method for controlling a computer-controlled medical apparatus, the power consumption of a gate control hardware module is limited, in particular to power consumption of less than 15 Win the case of a current intensity of less than 7 A.

For the security of the computer-controlled medical apparatus, it is particularly expedient if, in a method according to the invention for controlling this medical apparatus, security updates of software, in particular of the operating system, are exclusively applied to the gate control.

A method according to the invention for controlling this medical apparatus offers additional protection by virtue of an additional firewall implemented in hardware and/or software being used.

In a configuration of the method according to the invention for controlling a medical apparatus, network services which are provided by the gate control, in particular by the software of the gate control, are systematically minimized.

In a further configuration of the method according to the invention for controlling a medical apparatus, the gate control provides data services to secure the transfer of data via interfaces of the medical apparatus.

Another example embodiment includes a method for controlling a medical apparatus, in which the power supply of the gate control is derived from the power supply of the medical apparatus, more particularly by using Power over Ethernet (PoE).

### BRIEF DESCRIPTION OF THE DRAWINGS

The invention is explained in even greater detail below for example with reference to the accompanying figures, which also disclose features essential to the invention.

FIGS. 1a to 1c depict solutions of a computer controller for a computer-controlled medical apparatus according to the prior art, as already described above.

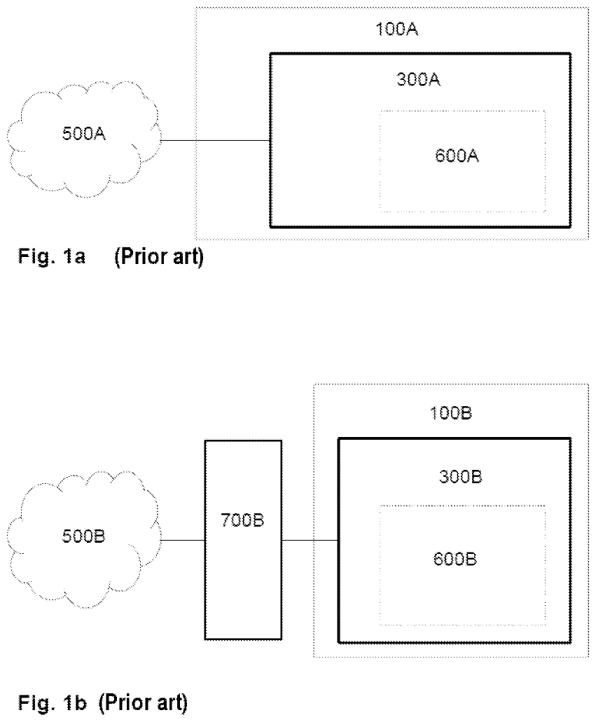

FIG. 2 depicts computer hardware according to an example embodiment of the invention for a computer-controlled medical apparatus for example representing represent an ophthalmological laser therapy system.

FIG. 3 depicts an ophthalmological laser therapy system.

### DETAILED DESCRIPTION

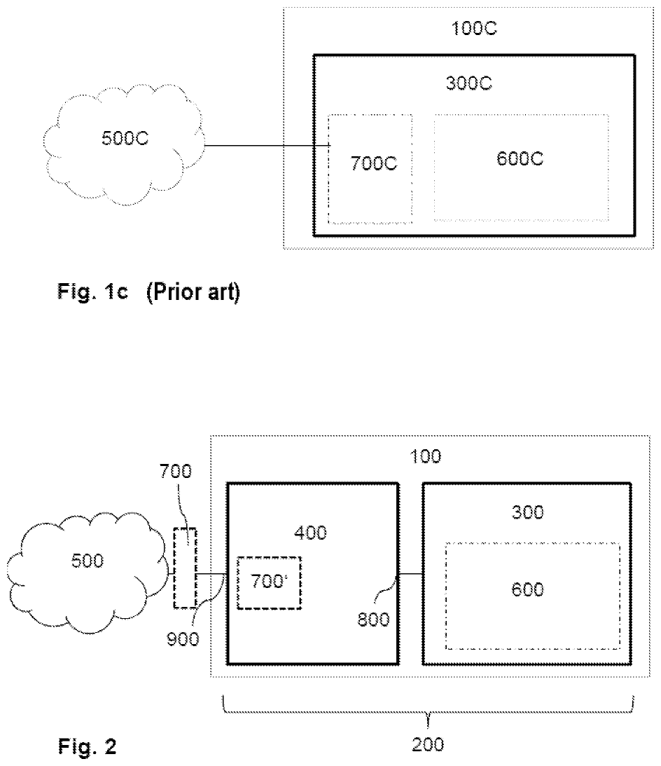

FIG. 2 shows computer hardware 200 according to the invention for a computer-controlled medical apparatus 100 which may in particular represent an ophthalmological laser therapy system 100 in this case. This computer hardware 200 comprises a control hardware module 300 serving an internal network 600 of the medical apparatus and a gate control hardware module 400 serving a communications network 500. In this case, the control hardware module 300 and the gate control hardware module 400 are disposed relative to one another such that there is a complete logical and in some embodiments also physical separation of communications network 500 and internal network 600 of the medical apparatus, and the gate control hardware module 400 forms a security entity between the communications network 500 and the internal network 600 of the medical apparatus 100. In this case, the gate control hardware module contains the interfaces 900 of the computer-controlled medical apparatus to the external communications network, that is to say the clinic communications network in this case. It furthermore contains interfaces 800 to the internal network 600 of the medical apparatus 100.

In this case, the control of the gate control hardware module 400 is exclusively implemented here from the internal network 600 of the medical apparatus 100, the latter being controlled by the control hardware module 300. Data required by the medical apparatus 100 are transmitted to the internal network 600 of the medical apparatus 100 by way of proprietary protocols.

Moreover, the computer hardware 200 according to the invention optionally contains a firewall 700, 700′, which, formed in terms of hardware as an external firewall 700, is placed in front of the gate control hardware module and/or which is represented by way of appropriate software in the gate control hardware module 400 as an internal firewall.

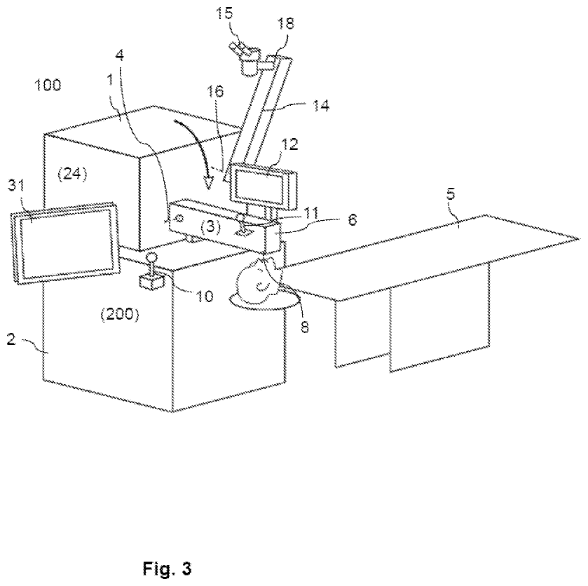

FIG. 3 describes an ophthalmological laser therapy system 100, that is to say an exemplary specific computer-controlled medical apparatus, in more detail in order to show how comprehensive the medically relevant functions of such a medical apparatus, in particular a medical apparatus having therapeutic functions, are.

The exemplary ophthalmological laser therapy system 100 attains a correction of the visual acuity of a patient's eye by application of a laser system, in particular by application of a pulsed and focused femtosecond laser beam, whose focal point separates tissue within the eye, in particular within the cornea and/or the lens of the patient's eye. By way of example, the refractive error can be corrected by separating a narrow lenticule in the cornea and subsequently extracting the lenticule via a small opening in the cornea.

The ophthalmological laser therapy system 100 of FIG. 3 is distinguished by a laser pivot arm 3 that is fastened to the apparatus head 1 in a manner pivotable about a horizontal axis 4 and that can be pivoted back and forth between a rest position and a work position. Since this laser pivot arm 3 is pivoted over the patient for laser therapy on the patient's eye but can be returned into a rest position in steps in which the laser pivot arm 3 is not required in order to use the space above the work position differently, the laser pivot arm 3 is protected by virtue of the laser pivot arm 3 being enclosed by a pivot arm housing 6, which is fastened in a pivotable manner to the apparatus head 1 in coaxial fashion in relation to the laser pivot arm 3 (“arm-in-arm principle”).

The pivot arm housing 6 has a laser exit opening 8 which is positioned such that, when the laser pivot arm 3 is brought into the work position, said laser exit opening is directed at the eye of a patient to be treated, who can be placed and positioned appropriately on the patient couch 5.

In this case, the ophthalmological laser therapy system 100 is composed of an apparatus base 2 and an apparatus head 1 that is adjustable on this apparatus base 2 in terms of its height above a plane of the floor, that is to say the z-direction, and in terms of its position in the plane, that is to say in the x- and y-directions. The apparatus head 1 contains a first part of the laser therapy optical unit required for performing the laser therapy. In the exemplary embodiment, the apparatus head 1 also contains the laser source, in this case a femtosecond laser source, required to produce a corresponding pulsed laser beam.

The second part of the laser therapy optical unit is rotatably mounted about a horizontal first axis 4 in a laser pivot arm 3. The laser pivot arm 3 can be pivoted about this first axis 4 from a rest position, in which it projects upward in approximately perpendicular fashion, into a work position, in which it is arranged approximately horizontally on the apparatus head 1, i.e., approximately parallel to the ground plane, and back again.

If use is furthermore made of a further independent examination pivot arm 14 in addition to the laser pivot arm 3, said examination pivot arm containing a surgical microscope 15 for example, the said examination pivot arm is arranged in a pivotable manner about an axis such that all work steps of a laser therapy on the eye of a patient can be carried out in such a way that the point of action of all work steps aided by the laser pivot arm 3 or the examination pivot arm 6 always remains stationary, that is to say the position of the patient's eye need not be changed during the entire laser therapy. The position of the patient's eye in the first work step determines the position of all subsequent work steps and hence the point of action of the devices thereof required in each case, which are on the various pivot arms 3, 14. This is facilitated by a special arrangement 300 of the pivot axes 4, 16 of the various pivot arms 3, 14 in relation to one another on the apparatus head 1 of the ophthalmological laser therapy system 100, wherein the first axis 4 of the laser pivot arm 3 and the second axis 16 of the examination pivot arm 14 have an appropriate arrangement 300 with respect to one another on the apparatus head 1, and, firstly, a therapy visual display unit 12 fastened to the pivot arm housing 6 in movable fashion is coupled to the movement of the pivot arm housing 6 and, secondly, the surgical microscope 15, which is movably fastened to the examination pivot arm 14, is coupled to the movement of the examination pivot arm 14 so that the therapy visual display unit 12 and the surgical microscope 15 always remain without tilt.

In this case, depending on the therapy step, one of the two arms, that is to say either the laser pivot arm 3 or the examination pivot arm 14, is situated so as to be pivoted over the patient in the work position and the other one of the two arms is in the rest position (folded up). The entire ophthalmological laser therapy system 100 is controlled here by computer hardware 200 according to the invention as described in FIG. 2, which is disposed in the interior of the ophthalmological laser therapy system 100 and which comprises a plurality of modules that are spatially and functionally separated from one another.

A typical course of the treatment is described below, for example as may be implemented for the separation of a lenticule in the cornea of a patient's eye and the subsequent extraction of the lenticule through a narrow opening, using an above-described ophthalmological laser therapy system 100:

First, the treatment or therapy parameters are planned on a planning visual display unit 31, which is likewise arranged directly on the ophthalmological laser therapy system 100 in this exemplary embodiment. However, alternatively, the planning visual display unit 31 may also be spatially separated from the ophthalmological laser therapy system 100. When planning, the ophthalmological laser therapy system 100 is for example in a standby position, that is to say the laser pivot arm 3 and optionally the examination pivot arm 14, too, are pivoted up vertically in the rest position on the system.

The patient is positioned on the patient couch 5. This is possible with some comfort on account of the pivoted-up laser pivot arm 3.

Then, the surgeon positions the height of the apparatus head 1 by manipulation of a joystick 10 on this apparatus head 1, by operation of which the translational movement of the apparatus head 1 over the apparatus base 2 can be controlled. In the process, orientation is provided by the image supplied by the camera 9, said image, including an overlaid symbol of a pivoted-down laser pivot arm 3, being visible on the therapy visual display unit 12 and/or on the planning visual display unit 31. As an alternative to the joystick, the positioning can also be effectuated in other embodiments by inputs on one of the two visual display units 12, 31 or by way of pushbuttons on the laser therapy system 100.

The surgeon triggers the motor-driven pivoting-down of the laser pivot arm 3 in, and together with, its pivot arm housing 6; a corresponding pushbutton employed to this end is not illustrated in the figures. As a result of the pre-positioning and the still retracted laser exit opening 8 of the laser pivot arm, a clear space remains between the laser exit opening 8 and the patient's eye, said clear space expediently having a size of between 50 mm and 150 mm.

Now, a contact glass is placed on the laser exit opening 8, if this has not yet happened in the rest position of the laser pivot arm 3. The contact glass is held against the laser exit opening 8 by application of a negative pressure. Activation and deactivation of the hold by application of a negative pressure is carried out in this case by virtue of pressing the contact glass against the laser exit opening 8; in the process, the latter is still slightly moved in its retracted position and the switching process is triggered. This example embodiment is advantageous over previously conventional laser therapy systems: There, the hold of the contact glass is switched separately. Consequently, the contact glass falls down when it is detached. By contrast, in the solution described here, the surgeon or operator always holds sway over the contact glass during the switching process.

Then, the surgeon initiates the release of the movement of the laser pivot arm 3 within the pivot arm housing 6 by application of a joystick rotation of the joystick 11 on the pivot arm housing 6, or alternatively by operation of a separate pushbutton (not illustrated). An automatic trigger of the movement by way of the applied contact glass is also possible in other embodiments. The laser exit opening 8 with the contact glass moves toward the eye in the process.

Finally there is the docking phase, that is to say the phase in which the contact glass is affixed: Here, the surgeon steers the contact glass toward the eye of the patient using the joystick 11 under observation by use of the video microscope 13. Fixating the eye by suctioning the eye to the contact glass is triggered by a pushbutton on the joystick 11 once the correct position has been reached. In one configuration, it is possible to assist the correct positioning or centering of the contact glass or another patient interface on the eye by virtue of processing the video microscope image and using the latter to control the apparatus head 1.

Hence, it is now finally possible by operation of a foot switch, which is not illustrated here, to start the actual laser therapy step by activating the laser beam, which is guided through the laser therapy optical units and the laser exit opening and focused in the patient's eye.

After completing this laser therapy step, the suctioning of the eye by application of the negative pressure is released by virtue of the pressure being increased here again, the laser pivot arm 3, and hence also the laser exit opening 8, are pivoted back into the pivot arm housing 6 again and the apparatus head 1 is slightly raised by a displacement in the z-direction. Hence, a safe distance from the eye is present once again. The contact glass or the patient interface can be removed from the laser exit opening 8, with the release being effectuated by brief upward pressure.

Now, the laser pivot arm 3 is pivoted up again together with its pivot arm housing 6; the clear space above the patient is re-established. Now, it is possible to perform further work steps or the patient can leave their position on the patient couch 5. The laser pivot arm 3 with its pivot arm housing 6 pivoting up is initiated electronically, by pushing a button in this case. Alternatively, the laser pivot arm 3 with its pivot arm housing 6 can be pushed manually until this is recognized by a position sensor on the pivot arm housing; following this, a motor takes over the movement.

However, where both eyes of a patient are to be treated, the apparatus head 1 can be moved by a translational movement in the x- and/or y-direction over the apparatus base 2 prior to pivoting-up of the laser pivot arm 3 with its pivot arm housing 6 in its rest position such that the laser pivot arm 3 with its pivot arm housing 6 is positioned over the other eye. A treatment of the second eye can then be effectuated in the same way by virtue of a new contact glass or patient interface being secured on the laser exit opening 8 by application of a negative pressure, and all steps following this being carried out as described above.

Furthermore, an examination pivot arm 14 containing an examination device, in this case a surgical microscope 15, is also fastened in a pivotable manner about a second axis 16 on the apparatus head 1 in this exemplary embodiment of an ophthalmological laser therapy system 100 according to the invention. By way of example, such a surgical microscope is required, or at least suggested, for the second main work step of the “SMILE” treatment. To this end, after pivoting up the laser pivot arm 3 with its pivot arm housing 6 into its rest position after completing the actual laser therapy step, as described here, the treatment is continued as follows:

The surgeon initiates the motor-driven downward pivoting of the examination pivot arm 14 by pressing a button. The motor moves the examination pivot arm 14 into its work position, where it rests on a stop. The work position is determined by expedient selection of the relative position of the two pivot axes, that is to say the first axis 4 and the second axis 16, and the end position of the examination pivot arm 14 that is determined by the stop is determined in such a way that the eye to be treated further lies in the examination volume of the surgical microscope 15 directly after pivoting down the examination pivot arm 14.

Minor corrections, to the extent that these are necessary, are possible by adjusting the position of the apparatus head 1 in relation to the apparatus base 2 by application of translational movements. Serving to this end is the joystick 10 present on the apparatus base 2, a separate foot console or a joystick present on the surgical microscope 15.

Once the examination pivot arm 14 with the surgical microscope 15 has been positioned accordingly, the lenticule extraction is carried out by the surgeon.

After completing the lenticule extraction, the examination pivot arm 14 with the surgical microscope 15 is pivoted up in a motor-driven manner and consequently pivoted back into its rest position. This can be initiated by pressing a button or else—as already described above for the pivot arm housing 6 and the laser pivot arm 3—by pushing. Hence, the clear space over the patient is re-established.

Most of these work steps harbor risks for the patient if the controller of the internal network 600 of the ophthalmological laser therapy system 100 has been corrupted: Changes in position may lead to bruising in the patient's eye, the fixation of the patient's eye on the contact glass may be released during the therapy, the use of the therapy laser beam may also be implemented at a different location to what was planned in the case of an incorrect transmission of the characterization data of the eye or in the case of an error-afflicted transmission of control data of the ophthalmological laser therapy system, incorrect measurement values may lead to incorrect therapy implementations. Therefore, all these functions are now separately protected by the computer hardware according to the invention and by a corresponding method for controlling a computer-controlled medical apparatus, as described above.

The aforementioned features of the invention, which are explained in various exemplary embodiments, can be used not only in the combinations specified in an exemplary manner but also in other combinations or on their own, without departing from the scope of the present invention.

A description of a piece of equipment relating to method features is analogously applicable to the corresponding method with respect to these features, while method features correspondingly represent functional features of the equipment described.

1.-19. (canceled)

20. Computer hardware for a computer-controlled medical apparatus, comprising:
a control hardware module serving an internal network of the medical apparatus; and
a gate control hardware module serving a communications network;
the control hardware module and the gate control hardware module being disposed such that there is a complete logical and physical separation of the communications network and the internal network of the medical apparatus, and
the gate control hardware module forming a security moderator between the communications network and the internal network of the medical apparatus.

21. The computer hardware as claimed in claim 20, wherein the computer hardware is configured to operate the control of the gate control hardware module exclusively from the internal network of the medical apparatus.

22. The computer hardware as claimed in claim 20, wherein the computer hardware is configured to forward required data to the internal network of the medical apparatus via proprietary protocols.

23. The computer hardware as claimed in claim 20, wherein power consumption of the gate control hardware module is limited.

24. The computer hardware as claimed in claim 23, wherein power consumption the power consumption of the gate control hardware module is limited to being less than 15 W or is limited to a current flow of less than 7 A.

25. The computer hardware as claimed in claim 20, wherein the computer hardware is configured to apply security updates to the software of the gate control hardware module.

26. The computer hardware as claimed in claim 20, further comprising a firewall implemented in hardware, in software or in both the hardware and the software.

27. The computer hardware as claimed in claim 20, wherein the computer hardware is configured to secure interfaces of the medical apparatus for the transfer of data by data services provided by the gate control hardware module.

28. The computer hardware as claimed in claim 20, wherein the computer hardware is configured to derive the power supply of the gate control hardware module from the power supply of the medical apparatus.

29. The computer hardware as claimed claim 20, wherein the gate control hardware module is disposed outside of the medical apparatus and comprises a mechanical protection for the control hardware module of the medical apparatus.

30. A computer-controlled medical apparatus, comprising computer hardware as claimed in claim 20.

31. A computer-controlled ophthalmological laser therapy system, comprising computer hardware as claimed in claim 20.

32. A method of controlling a computer-controlled medical apparatus, comprising:
exclusively operating medically relevant software on an internal network of the medical apparatus;
exclusively communicating between the medical apparatus and an external communications network by operation of a gate control module;
securing the medical apparatus vis-à-vis the external communications network by operation of the gate control module by admitting no data traffic between the internal network and the gate control module, and hence admitting no data traffic to the external communications network, until there is a request from the internal network or a qualified manual input by an operator, and
implementing with the gate control module a complete logical and physical separation of communications network and internal network of the medical apparatus.

33. The method as claimed in claim 32, further comprising implementing the control of the gate control module exclusively from the internal network of the medical apparatus.

34. The method as claimed in claim 32, further comprising transmitting data required by the medical apparatus to the internal network of the medical apparatus via proprietary protocols.

35. The method as claimed in claim 32, further comprising limiting power consumption of a gate control hardware module.

36. The method as claimed in claim 35, further comprising limiting the power consumption of the gate control hardware module to less than 15 W or to a current flow of less than 7 A.

37. The method as claimed in claim 32, further comprising exclusively applying security updates of software or to an operating system to the gate control module.

38. The method as claimed in claim 32, further comprising implementing an additional firewall in hardware, in software or both in the hardware and the software.

39. The method as claimed in claim 32, further comprising systematically minimizing network services which are provided by the gate control or by the software of the gate control.

40. The method as claimed in claim 32, further comprising securing transfer of data services via interfaces of the medical apparatus by application of the gate control module.

41. The method as claimed in claim 32, further comprising deriving power supply of the gate control from the power supply of the medical apparatus, more particularly by using Power over Ethernet (PoE).

42. The method as claimed in claim 41, further comprising deriving power supply of the gate control from the power supply of the medical apparatus by using Power over Ethernet (PoE).
